(19) United States
(12) Patent Application Publication
(10) Pub. No.: US 2010/0170151 A1
(43) Pub. Date: Jul. 8, 2010

(54) Aquaculture Harvesting, Gas Exchange, and Media Circulation Device and Method of Use

(76) Inventor: Matthew Peter Huber, Carlsbad, CA (US)

Correspondence Address: ERIC HANSCOM, 7395 PORTAGE WAY, CARLSBAD, CA, 92011, US

(21) Appl. No.: 12/684,238

(22) Filed: Jan. 8, 2010

Related U.S. Application Data
(60) Provisional application No. 61/143,208, filed on Jan. 8, 2009.

(51) Int. Cl. A01H 13/00

(52) U.S. Cl. 47/1.4

(57) ABSTRACT

An aquaculture harvesting, gas exchange, and media circulation device and method of use for the cultivation and collection of microalgae is disclosed. The current invention couples water motion, gas exchange, and harvesting into one comprehensive system. A modified airlift pump is used to circulate water, exchange gas, and facilitate harvesting. By eliminating other energy consuming components, a more energy efficient and cost effective device and method is disclosed for the production of microalgae, which can be used as a biofuel or food source while simultaneously removing carbon dioxide from the environment.

### CROSS REFERENCE TO RELATED APPLICATIONS

This application is a non-provisional application of Provisional Patent Application No. 61/143,208 filed on Jan. 8, 2009, the entirety of which is hereby incorporated by reference.

### STATEMENT REGARDING FEDERALLY SPONSORED RESEARCH OR DEVELOPMENT

This invention was not federally sponsored.

### BACKGROUND OF THE INVENTION

### Field of the Invention

This invention relates to the general field of aquaculture, and more specifically toward an aquaculture harvesting, gas exchange, and media circulation device and method of use for the cultivation and collection of microalgae. The current invention couples water motion, gas exchange, and harvesting into one comprehensive system. A modified airlift pump is used to circulate water, exchange gas, and facilitate harvesting. By eliminating other energy consuming components, a more energy efficient and cost effective device and method is disclosed for the production of microalgae, which can be used as a biofuel or food source while simultaneously removing carbon dioxide from the environment.

In aquaculture, water motion is often manipulated by the use of a paddle wheel. This method can be expensive and requires frequent maintenance as it involves moving parts that are in contact with water. The paddlewheel operates by rotating a series of blades extended from a center axel. The axel is turned by a gearbox and motor. The resultant force on the water generates a current, which, depending on the desired specifications, can reach speeds of up to 50 centimeters per second (1.64 feet per second).

Gas exchange in aquaculture pond systems is often accomplished by diffusing a gas of interest into the water. Commonly, oxygen is bubbled into aquaculture systems where heterotrophic organisms are the product and carbon dioxide where autotrophic organisms are the product. Atmospheric gas (air) is commonly bubbled into water for this purpose, as it contains both gases. Air is a preferred gas because most organisms have evolved at equilibrium with gas concentrations in the air, and it is widely available when taken from the atmosphere. However, the atmosphere has relatively low concentrations of oxygen and carbon dioxide, thus it is often not profitable and/or at least physically difficult to bubble extremely large volumes of air into a pond system to maintain consistent concentrations of target gases. Operators often choose to bubble oxygen or carbon dioxide into their ponds rather than bubble air, as the total volume of gas is smaller, allowing for a smaller gas diffusion system. The purchase of oxygen or carbon dioxide adds to the total cost of the operation. A significant issue exists in the fact that carbon dioxide gas available for purchase is likely of ‘fossil’ origin, such as from coal, natural gas, or oil. Using carbon dioxide gas from ‘fossil’ sources does no draw down atmospheric concentrations of carbon dioxide. The carbon source question becomes central when the goal is to use an algae pond system to generate a carbon-neutral or carbon-negative biofuel.

In aquaculture, the separation of water from microorganisms, also called dewatering or harvesting, can be accomplished in many ways. Some of the most commonly used methods include: centrifugation, size separation (filtration), flocculation, positive or negative density separation, and foam/froth separation. All of these methods can be effective at separating water from microorganisms including microalgae. Selection of a harvesting device involves considering the target species, complying with regulatory codes, and energy/cost expenditures per unit of product. Foam or froth harvesting has been shown to be effective at concentrating algae.

U.S. Pat. No. 4,707,308 to Ryall discloses an airlift pump in an open bottom cone to aerate and circulate water in a pond, moving water only in an upward, non-directional manner. It does not use air bubbles to directionally advect water thereby creating a current.

U.S. Pat. No. 3,768,200 to Klock teaches a system used to treat wastewater and uses the products to produce algae. Klock describes using sedimentation flocculation coupled to collection of microalgae in filter bags as the harvesting method. This system is inferior because bags, filtering, and/or flocculation are used. Filtering requires time and manpower to clean and replace filters. Flocculation requires close human management of microalgae culture to control and predict sedimentation. An airlift pump is used to perform gas exchange and to create a u-turn in the current. However, Klock does not disclose or teach a single system that creates currents in pond systems, promotes gas exchange, and collects microalgae in foam. The teachings of Klock cannot propel water in a straight line or at an angle and requires the water to be moved underground in pipes.

U.S. Pat. No. 5,910,254 to Guelcher et al. describes a complex multistage dewatering system for the concentration of microalgae. It requires a complex system to collect algae suspensions and is inferior for larger algae culture systems since it does not combine the dewatering process with water motion and gas exchange.

U.S. Pat. No. 6,524,486 to Borodyanski et al. discloses a telescoping column for foam collection followed by cloth filtration and a drying unit. In fact, it requires a complex column system to collect algae suspensions. It is inferior for large algae culture systems because it does not combine the dewatering process with water motion and gas exchange.

Thus there has existed a long-felt need for a device and method to cultivate and collect microalgae that is at least carbon-negative and preferably carbon neutral, where the water motion, gas exchange, and harvesting required by the method are integrated into one comprehensive system that is both energy efficient and cost effective.

### SUMMARY OF THE INVENTION

The current invention provides just such a solution by having an aquaculture harvesting, gas exchange, and media circulation device and method of use for the cultivation and collection of microalgae. The current invention couples water motion, gas exchange, and harvesting into one comprehensive system. A modified airlift pump is used to circulate water, exchange gas, and facilitate harvesting. By eliminating other energy consuming components, a more energy efficient and cost effective device and method is disclosed for the production of microorganisms such as microalgae, which can be used as a biofuel or food source while simultaneously removing carbon dioxide from the environment.

The device, system, and method of the current invention use a modified airlift pump to create a water current. Air is introduced on one side of a u-shaped bend in the path of the water across the entire width of the pond. Gas exchange is achieved by releasing large volumes of gas into the airlift pump through appropriate diffusers. The harvesting of a product is achieved by collecting foam made from the air bubbles as they reach the water's surface. The current invention combines water advection, gas exchange, and harvesting into one device. Gas used in the gas exchange is pulled from the atmosphere rather than from sources derived from ‘fossil’ fuels.

By using the device and method of the current invention, users will save money for many reasons. There will be no need to pump water to a centralized harvesting plant. Further, paddle wheels or other means to agitate and/or circulate the water are not required. Since air from the atmosphere is used, additional or specialized gasses are not needed. An additional gas exchange system, separate from the means of circulating the water, is not necessary. Because there are fewer required components and materials, the overall device of the current invention is cheaper to manufacture and operate.

It is a principal object of the invention to provide a device and method that is at least carbon neutral and preferably carbon-negative when the device is employed to cultivate carbon-fixing organisms.

It is another object of the invention to provide a cost effective device and method for removing carbon dioxide from the atmosphere.

It is an additional object of this invention to provide an efficient means of producing algae for use with biofuels.

It is a further object of this invention to provide an efficient means of producing organisms that directly or indirectly feed on microalgae, for use as a feed or with biofuels.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims.

### BRIEF DESCRIPTION OF THE FIGURES

The accompanying drawings, which are incorporated in and form a part of this specification, illustrate embodiments of the invention and together with the description, serve to explain the principles of this invention.

FIG. 1 is a perspective cutaway view of the aquaculture harvesting, gas exchange, and media circulation device according to the current invention.

FIG. 2 is a perspective upstream side view of the device.

FIG. 3 is a perspective side view of the device.

FIG. 4 is a perspective top view of the device.

FIG. 5 is a perspective front cutaway view of the device in operation.

FIG. 6 is a perspective downstream view of the device in operation.

FIG. 7 is a top view of the device operating in a racetrack configuration.

### DETAILED DESCRIPTION OF THE INVENTION

Many aspects of the invention can be better understood with the references made to the drawings below. The components in the drawings are not necessarily drawn to scale. Instead, emphasis is placed upon clearly illustrating the components of the present invention. Moreover, like reference numerals designate corresponding parts through the several views in the drawings.

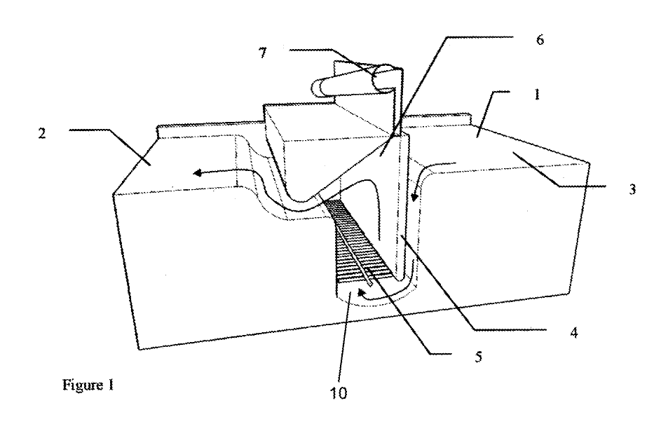

FIG. 1 is a perspective cutaway view of the aquaculture harvesting, gas exchange, and media circulation device according to the current invention. Water, the flow of which is shown by the directional arrows throughout the figures, flows over the pond floor 3 from the upstream side 1 to the downstream side 2. The water flows into a depression 10 around a divider 4. The divider 4 is a barrier that forces the path of flowing water down one side of the depression 10 and up the other. The divider 4 begins above the waterline and extends below the water line, but does not extend to the bottom of the depression 10, thereby allowing water to pass underneath the divider 4. After passing around the divider 4 to the downstream side 2, the water passes through a plurality of gas diffusers 5. The plurality of gas diffusers 5 release bubbles 8 into the flow of water. The bubbles entrain the water and force it to the surface, in a similar fashion to that of an air lift pump. The resultant water level on the downstream side of the device is higher than on the upstream side, promoting the water advection by gravity.

In addition to moving the water from the upstream side to the downstream side, the gas diffusers 5 promote gas exchange within the system. As stated above, the preferred source of gas for the gas exchange is air; it is inexpensive and also helps draw down the amount of carbon dioxide in the atmosphere. The flowing water with diffused gases is an ideal medium for the growth of algae. The plurality of gas diffusers could be one or more devices that include a plurality of openings that allow gas to bubble into water.

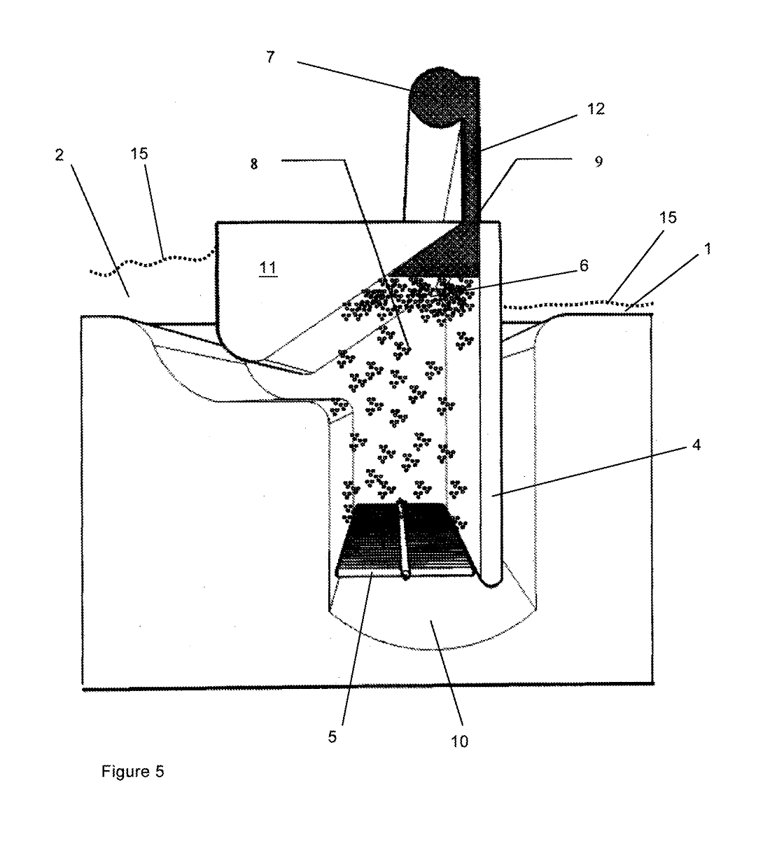

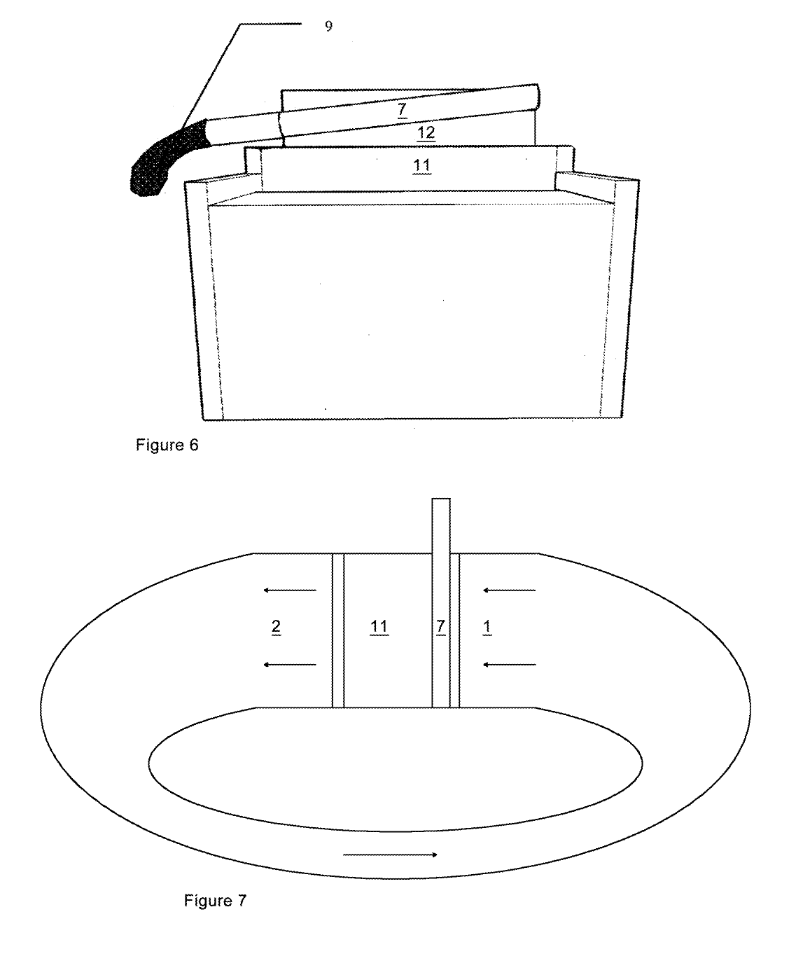

Harvesting is accomplished by placing a semi-enclosed foam collection chamber 6 above the gas diffusers 5 in such a way as to collect the foam (shown in FIGS. 5 and 6) while still allowing water to pass downstream. A collection chamber barrier 11 extends below the water's surface thereby preventing foam from escaping the foam collection chamber 6. The foam, which contains the algae product, is forced up and out of the foam collection chamber 6 into the foam conduit 7 by the continuous formation of more foam. The foam floats out of the foam collection chamber 6, through a foam collection channel 12, and into the foam conduit 7, where the foam conduit 7 is preferably a downward angled tube that facilitates the movement of the foam away from the device by using gravitational forces. The foam is then passed from the foam conduit 7 to a defoaming area where it is melted into a concentrated liquid product. The concentrated liquid product can then be modified for use as fuel, food, or other algae containing products.

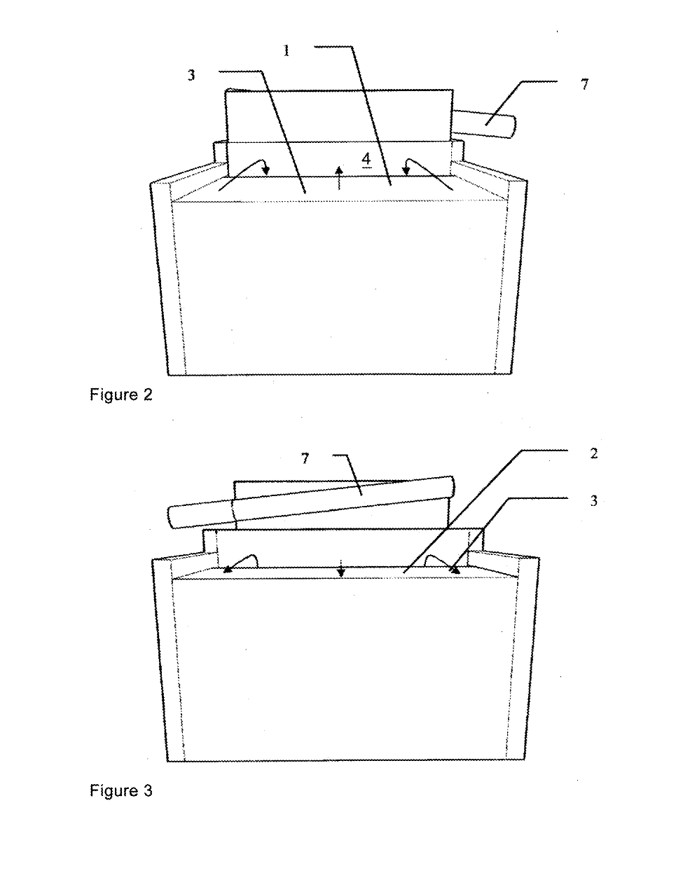

FIG. 2 is a perspective upstream side view of the device. The water travels over the pond floor 3 of the upstream side 1 into the divider 4, which then forces the water into the depression. The foam conduit 7 is shown extending out of the backside of the device; however, it could just as easily extend out of the front or any other direction of the device that would be beneficial for the implementation of the current invention.

FIG. 3 is a perspective downstream side view of the device. The water travels out of the depression to the downstream side 2 over the pond floor 3.

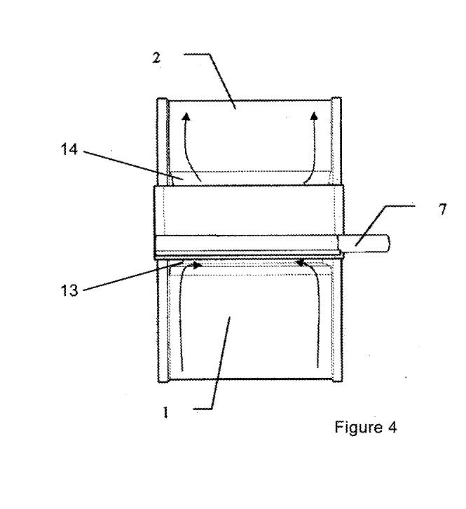

FIG. 4 is a top view of the device. The water travels from the upstream side 1 into the upstream side of the depression 13, out the downstream side of the depression 14, and out the downstream side 2.

FIG. 5 is a perspective front cutaway view of the device in operation. As the water flows through the depression, the gas diffusers 5 diffuse gas into the water in the form of bubbles 8. These bubbles apply a force to the water causing the water to move upward. This upward force helps create the flow of water through the device, even if the pond floor 3 is completely level throughout the system. The bubbles 8 travel upward and collect in the foam collection chamber 6 formed by the collection chamber barrier 11. The resulting foam 9 is formed at the top of the foam collection chamber 6 and is forced into the foam collection channel 12 and into the foam conduit 7. The water, substantially free of bubbles, foam, and algae, travels around the collection chamber barrier 11 and out of the depression 10 into the downstream side 2. The water level immediately downstream is higher than the water level immediately upstream, though the difference between the two may be very small.

FIG. 6 is a perspective downstream view of the device in operation. Foam 9 travels out of the foam conduit 7 to a defoaming area where it is melted into a concentrated liquid product. The concentrated liquid product can then be modified for use as fuel, food, or as a part of other algae containing products.

FIG. 7 is a top view of the device operating in a racetrack configuration. The water level immediately downstream is higher than the water immediately upstream. When the system using the current device is formed into a closed system, such as a racetrack shape shown in FIG. 7, the water flows from the downstream side back around to the upstream side. In this embodiment, the device includes a return that allows the water to flow from the downstream side back to the upstream side such that it can continuously flow through the device. Therefore, the entire flow of water is maintained by the upward movement of the bubbles produced by the gas diffusers.

The production of algae requires carbon dioxide and produces oxygen. Therefore, if the gas used in the device is air, the production of algae using this device can be carbon negative. The net amount of carbon removed from the air using the current invention depends on, among other things, the energy required to operate the device, the size of the device itself, and the amount, quality, and type of algae produced.

The collection chamber barrier extends below the downstream water level such that the bubbles and resulting foam collect in the foam collection chamber. The shape of the collection chamber barrier is preferably that of a partial airfoil. This helps reduce turbulent flow through the depression and promotes the transition of water flow moving in an upward direction to water flow moving in a lateral direction. The foam collection chamber need not be completely enclosed; although, it is a preferred embodiment as wind may blow foam away from the designated area.

The shape of the depression is also preferably formed to promote the laminar flow of water through the device. While the device is shown with open means of transporting the water upstream and downstream, it is nonetheless possible to use enclosed pipes to transport the water to the upstream side of the device and away from the downstream side of the device.

The gas diffuser uses a means of producing air pressure to force a gas through the gas diffusers. This means of producing air pressure can be a low-pressure air blower that consumes a relatively little amount of energy during use. The gas, preferably air, can be collected from the atmosphere. Instead of using a low-pressure air blower, it is also possible to use an air intake system of appropriate shape and size that uses the natural force of the wind to cause the air to travel through the gas diffusers and into the water.

Algae culturing is preferably formed using a water velocity within the range of 20 to 50 centimeters per second. This is achieved by manipulating the volume of gas forced through the gas diffusers, the size of the air bubbles, and the size and shape of the depression. Generally, in mass culture, twice the mass of carbon contained in a gas is needed to satisfy the mass of carbon demand by microalgae. The atmosphere contains roughly 0.5 grams CO₂ per cubic meter. Accordingly, a pond measuring 10 cubic meters in volume would demand 1,000 grams of CO₂ per day resulting in a requirement of 2,000 cubic meters of air to be bubbled per day. In other words, 15 percent of the pond's volume should be bubbled per minute.

The current invention provides the same result as a protein skimmer, whereby a physical structure is used to collect and remove aquaculture products; however, the result is achieved in a new and novel fashion.

The device and method of the current invention harvests foam commonly seen in aquaculture. The cause of foam formation in aquaculture results from gas entrapment in a solution that acts to form foam, which includes without limitation: dissolved organic matter (DOM), dissolved organic carbon (DOC), DOM and DOC released from microorganisms, and/or addition of foaming agents. Dissolved organic matter is intended to include microalgae, or simply algae.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

What I claim is:

1. A device for the collection of aquaculture products comprising
a divider, a depression, a plurality of gas diffusers, a collection chamber, and water,
where the water travels around the divider and into the depression, where the plurality of gas diffusers emit a gas into the water, where the gas bubbles upwards into the collection chamber, whereby aquaculture products can be harvested from the collection chamber.

2. The device of claim 1, further comprising a collection chamber barrier, where the collection chamber barrier in part forms the collection chamber, where the collection chamber barrier is shaped such that water travels beneath it when leaving the depression.

3. The device of claim 1, further comprising a means of collecting foam.

4. The device of claim 3, wherein the means for collecting foam comprises a foam conduit, where foam is transported away from the device via the foam conduit

5. The device of claim 1, further comprising an upstream portion and a downstream portion, where water travels from the upstream portion into the depression, and where water travels from the depression to the downstream portion.

6. The device of claim 5, wherein the upstream portion comprises an upstream water level, wherein the downstream portion comprises a downstream water level, where the downstream water level is higher than the upstream water level.

7. The device of claim 5, further comprising a return, where the return connects the downstream side to the upstream side, whereby water flows from the downstream side to the upstream side using the return.

8. The device of claim 1, wherein the gas is air.

9. The device of claim 1, further comprising a low-pressure air pump, where the low-pressure air pump provides gas to the gas diffusers.

10. A method of collection of dissolved organic matter comprising the steps of
imparting an upward force to water by means of a plurality of gas diffusers, whereby water is drawn into a depression and flows around a divider and through the plurality of gas diffusers, where the plurality of gas diffusers create bubbles of gas that travel upward into a collection chamber and create a foam,
collecting the foam from the collection chamber, where the foam comprises dissolved organic matter.

11. The method of claim 10, wherein the gas is air.

12. The method of claim 10, wherein the water exits the depression around a collection chamber barrier.

13. The method of claim 10, wherein the foam is collected using a foam conduit.

14. The method of claim 10, further comprising the step of circulating water that exits the depression back into the depression.

15. The method of claim 10, wherein gas is provided to the means of plurality of gas diffusers by a low-pressure air pump.

16. The method of claim 10, wherein the flow of water through the depression is substantially laminar.

17. A device for collecting algae consisting essentially of
a pond, a depression, a plurality of gas diffusers, a divider, a collection chamber, and a means of collecting foam,
where the pond comprises an upstream portion and a downstream portion, where water flows from the upstream portion around a divider into the depression, where the plurality of gas diffusers is located within the depression, where the plurality of gas diffusers emit a gas into the water, where the gas bubbles upward and creates a foam in the collection chamber, where the means of collecting foam collects foam from the collection chamber, where the foam comprises algae.

18. The device of claim 17, wherein the gas is air.

19. The device of claim 17, wherein the collection chamber comprises a collection chamber barrier, where the water flows around the collection chamber barrier to the downstream portion.

20. The device of claim 17, wherein the means of collecting foam comprises a foam conduit, whereby foam travels out of the collection chamber and into the foam conduit, where the foam is transported away from the device via the foam conduit.
